(19) United States
(12) Patent Application Publication
(10) Pub. No.: US 2012/0330352 A1
(43) Pub. Date: Dec. 27, 2012

(54) TRANSAPICAL CLOSURE DEVICES AND METHODS FOR USE

(75) Inventors: Farhad Khosravi, Los Altos Hills, CA (US); Andy Uchida, Los Altos, CA (US); Jose Garcia, Fremont, CA (US); Brandon Fell, Dublin, CA (US)

(73) Assignee: AccessClosure, Inc., Mountain View, CA (US)

(21) Appl. No.: 13/531,534

(22) Filed: Jun. 23, 2012

Related U.S. Application Data
(60) Provisional application No. 61/501,135, filed on Jun. 24, 2011.

(51) Int. Cl. A61B 17/03

(52) U.S. Cl. 606/213

(57) ABSTRACT

Apparatus and methods are provided for sealing a puncture through the wall of a patient's heart into a heart chamber, e.g., at the apex of the heart into the left ventricle. A positioning member is advanced into the puncture until a positioning element thereon is disposed within the chamber, the positioning element is expanded within the chamber, and the positioning member is withdrawn until the expanded positioning element contacts the endocardial wall of the chamber. A cartridge carrying a solid sealant is advanced over the positioning member, and the sealant is deployed within the puncture, e.g., offset proximally from the endocardial wall, the sealant expanding upon exposure to fluid within the puncture to seal the puncture.

### RELATED APPLICATION DATA

The present application claims benefit of co-pending U.S. provisional application Ser. No. 61/501,135, filed Jun. 24, 2011, the entire disclosure of which is expressly incorporated by reference herein.

### FIELD OF THE INVENTION

The present invention relates generally to apparatus and methods for sealing punctures in tissue, and more particularly, to apparatus and methods for sealing a puncture or hole in the wall of a heart, e.g., a puncture in the ventricular wall of the heart.

### BACKGROUND

Apparatus and methods are known for accessing a patient's vasculature percutaneously, e.g., to perform a procedure within the vasculature, and for sealing the puncture that results after completing the procedure. For example, a hollow needle may be inserted through a patient's skin and overlying tissue into a blood vessel. A guide wire may be passed through the needle lumen into the blood vessel, whereupon the needle may be removed. An introducer, procedural, or femoral sheath may then be advanced over the guide wire into the vessel, e.g., in conjunction with or subsequent to one or more dilators. A catheter or other device may be advanced through the introducer sheath and over the guide wire into a position for performing a medical procedure. Thus, the introducer sheath may facilitate accessing and/or introducing various devices into the vessel, while minimizing trauma to the vessel wall and/or minimizing blood loss.

Upon completing the procedure, the device(s) and introducer sheath may be removed, leaving a puncture extending between the skin and the vessel wall. To seal the puncture, external pressure may be applied to the overlying tissue, e.g., manually and/or using sandbags, until hemostasis occurs. This procedure, however, may be time consuming and expensive, requiring as much as an hour of a medical professional's time. It is also uncomfortable for the patient, and may require the patient to remain immobilized in the operating room, catheter lab, or holding area. In addition, a risk of hematoma exists from bleeding before hemostasis occurs.

Various apparatus and methods have been suggested for sealing vascular punctures resulting from such procedures, such as those disclosed in U.S. Pat. Nos. 7,316,704, 7,331,979, 7,335,220, and 7,806,856, and U.S. Publication Nos. 2007/0231366, 2008/0082122, 2009/0088793, 2009/0254110, 2010/0168789, 2010/0274280, and 2010/0280546. The entire disclosures of these references are expressly incorporated by reference herein.

For example, the MATRIX™ product included two synthetic polyethylene glycol (“PEG”) polymer powders that were mixed with appropriate buffers and injected through a femoral sheath at an arteriotomy site, e.g., as disclosed in U.S. Pat. No. 7,316,704. The Mynx® Vascular Closure Device is another system for sealing vascular punctures, e.g., as disclosed in one or more of the references identified above, such as U.S. Pat. No. 7,335,220.

Accordingly, apparatus and methods for sealing a puncture through tissue would be useful.

### SUMMARY

The present invention is directed to apparatus and methods for sealing a puncture in tissue. More particularly, the present invention is directed to apparatus and methods for sealing a puncture in the wall of a heart, e.g., a puncture or hole in the ventricular wall of the heart.

In the continuing push toward minimally invasive medical procedures, treatments that formerly required open surgery are being performed by medical devices developed to treat target organs through small incisions that are more easily closed using sutures or other closure methods. Smaller incisions can potentially lead to fewer complications before and after the procedure.

For example, surgical procedures may be performed via an access hole in the ventricular wall of the heart. Typically, this approach is called a “transapical approach” since the device may be introduced through the apex of the left ventricle. However, similar surgical openings can be created for accessing other chambers of the heart as well as other organs of the body.

Conventional devices and methods for sealing vascular punctures may not be easily applicable to such openings through the wall of the heart. The openings involved are formed through a relative thick tissue body and may be substantially larger than conventional vascular punctures, e.g., in the femoral or other peripheral arteries of the body. Further, such openings may be more difficult to access than punctures through a patient's skin into an artery, since the heart must be accessed through an opening in the patient's chest, e.g., a sternotomy, thoracotomy, and the like.

In accordance with one embodiment, a method is provided for performing a medical procedure that includes providing access to a patient's heart; creating a passage through the myocardium of the heart into a chamber of the heart; introducing a distal end of an access sheath through the passage into the chamber of the heart; and performing one or more medical procedures via the access sheath. Upon completing the medical procedure(s), a sealant is introduced into the passage such that a distal end of the sealant is offset from the endocardial wall of the chamber by a predetermined offset distance, and the access sheath may be removed from the heart.

In an exemplary embodiment, the sealant may be introduced by inserting a tubular cartridge carrying the sealant into the access sheath, and withdrawing the cartridge and the access sheath to deploy the sealant within the passage, e.g., such that the distal end of the sealant is offset from the endocardial wall of the chamber by the predetermined offset distance, e.g., at least about two millimeters (2 mm).

For example, a positioning member may be introduced into the access sheath until a positioning element thereon is disposed within the chamber, the positioning element may be expanded within the chamber, and then the positioning member may be withdrawn until the expanded positioning element contacts the endocardial wall of the chamber adjacent the passage. The cartridge may then be inserted into the access sheath by advancing the cartridge over the positioning member. Optionally, the cartridge and the positioning member may include cooperating elements that limit advancement of the cartridge over the positioning member, e.g., such that the distal end of the sealant is offset by the predetermined offset distance.

Thereafter, the positioning element may be collapsed after deploying the sealant within the passage, and the positioning member may be withdrawn such that the collapsed positioning element passes through the sealant deployed within the passage.

In accordance with another embodiment, a method is provided for sealing a puncture through the wall of a patient's heart that includes introducing a positioning member into the puncture until a positioning element thereon is disposed within the chamber; expanding the positioning element within the chamber; and withdrawing the positioning member until the expanded positioning element contacts the endocardial wall of the chamber adjacent the passage. A cartridge carrying a solid sealant may be advanced over the positioning member, and the sealant may be deployed within the puncture, the sealant expanding upon exposure to fluid within the puncture to substantially seal the puncture.

In an exemplary embodiment, the cartridge may be advanced over the positioning member to position a distal end of the sealant spaced apart from the positioning element, thereby offsetting the sealant from the endocardial wall of the chamber by a predetermined offset distance. For example, the cartridge and the positioning member may include cooperating elements that limit advancement of the cartridge over the positioning member until the distal end of the sealant is offset by the predetermined offset distance.

In accordance with yet another embodiment, a method is provided for sealing a puncture through the wall of a patient's heart using an apparatus comprising an elongate positioning member carrying a cartridge, the cartridge carrying a sealant and a pusher member adjacent the sealant within a lumen of the cartridge. The method may include introducing a distal end of the positioning member into the puncture with the cartridge until a positioning element thereon is disposed within a chamber of the heart; expanding the positioning element within the chamber; and withdrawing the positioning member from the puncture until the expanded positioning element contacts an endocardial wall of the chamber adjacent the puncture. The cartridge may be advanced over the positioning member into the puncture until a distal end of the sealant is spaced apart from the positioning element, thereby offsetting the sealant from the chamber by a predetermined distance; and then the cartridge may be retracted to deploy the sealant within the puncture.

In accordance with still another embodiment, an apparatus is provided for sealing a puncture extending through the wall of a patient's heart that includes a cartridge comprising a proximal end, a distal end sized for insertion into a puncture, a lumen extending between the proximal and distal ends, and a distal opening in communication with the lumen; sealant disposed within the lumen, having a predetermined outer diameter before being deployed from the cartridge that is at least about two millimeters (2 mm); and an advancer or tamp member disposed within the lumen of the cartridge for deploying the sealant from the lumen out the distal opening when the cartridge is retracted from a puncture relative to the advancer member. In other embodiments, before being exposed to fluid, i.e., when provided in the cartridge before delivery, the sealant may have an outer diameter of at least about four millimeters (4 mm) or at least about six millimeters (6 mm), and a length between about five and thirty millimeters (5-30 mm), at least about two millimeters (2 mm), five millimeters (5 mm), ten millimeters (10 mm), and the like.

In one embodiment, the apparatus also includes an elongate positioning member having an expandable element on a distal end thereof, the positioning member sized for passing through the lumen of the cartridge and the sealant. For example, the cartridge, with the sealant and advancer member therein, may be provided initially in a proximal position on the positioning member, the cartridge being advanceable along the positioning member to a distal position wherein the sealant is disposed adjacent the positioning element. Optionally, the cartridge and the positioning member may include cooperating elements that limit advancement of the cartridge over the positioning member until the distal end of the sealant is offset by a predetermined offset distance, e.g., at least about two millimeters (2 mm).

Other aspects and features of the present invention will become apparent from consideration of the following description taken in conjunction with the accompanying drawings.

### BRIEF DESCRIPTION OF THE DRAWINGS

It will be appreciated that the exemplary apparatus shown in the drawings are not necessarily drawn to scale, with emphasis instead being placed on illustrating the various aspects and features of the illustrated embodiments.

FIG. 1 is a side view of an exemplary embodiment of an apparatus for sealing an opening in the wall of a heart.

FIGS. 2-4 are side views of the apparatus of FIG. 1, showing an exemplary method for sealing an opening in the apex of a left ventricle of a heart.

FIGS. 5-8 are cross-sectional details of the heart of FIGS. 2-4, showing additional details of an exemplary method for sealing an opening in the apex of the left ventricle of the heart.

### DETAILED DESCRIPTION OF THE EXEMPLARY EMBODIMENTS

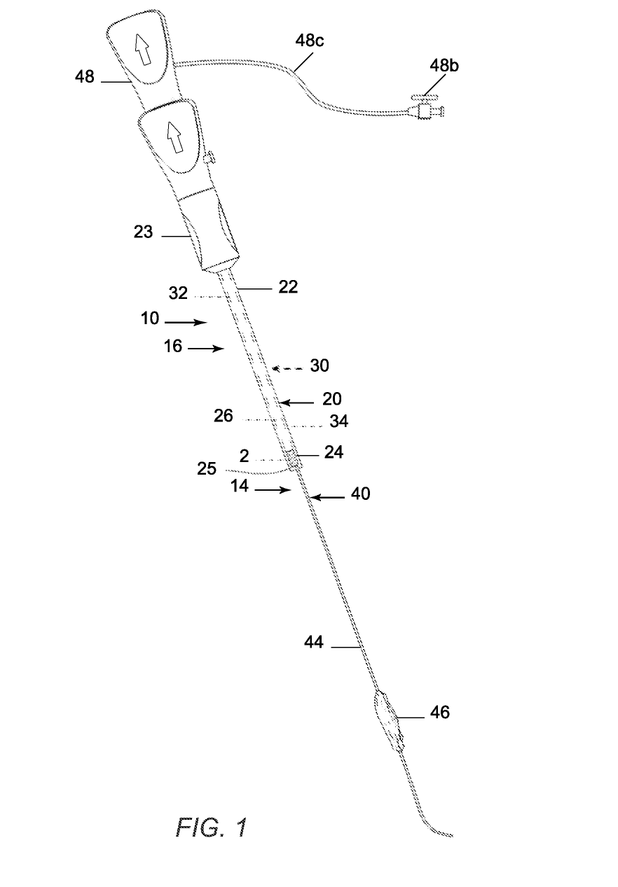

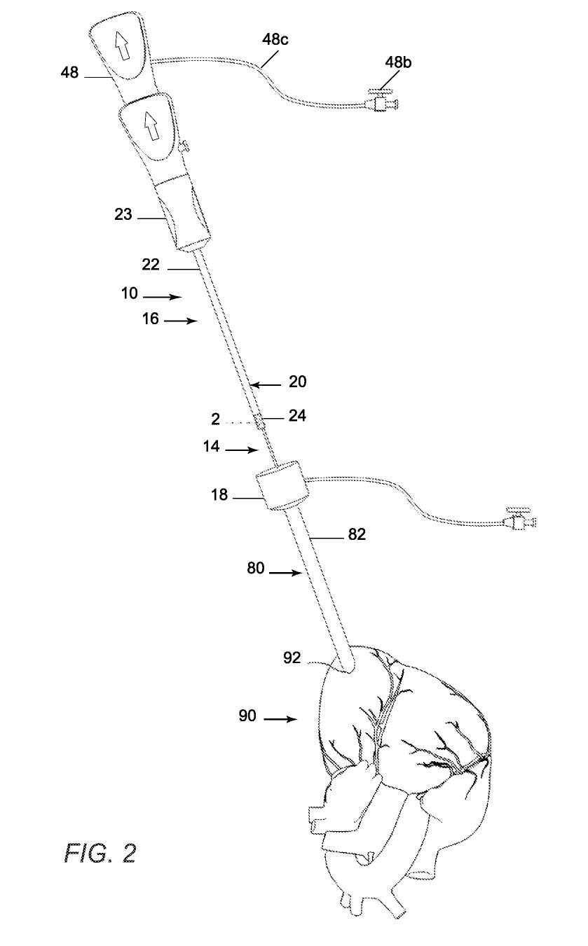

Turning to the drawings, FIG. 1 shows an exemplary embodiment of an apparatus 10 for sealing a puncture or opening through tissue, for example, an opening 92 through the wall 94 of a heart 90, e.g., for accessing the left ventricle 96, as shown in FIGS. 2-8. Generally, the apparatus 10 includes a positioning member 14 and a cartridge or shuttle 16 carried on the positioning member 14 for delivering a pellet or other sealant 2, which may be any of the embodiments described elsewhere herein or in the references incorporated by reference herein therein into a puncture (not shown). Optionally, the apparatus 10 may be part of a system, e.g., which may also include a delivery, access, procedure, introducer, or other sheath 80, e.g., as shown in FIGS. 2-4. Optionally, the apparatus 10 and/or system may include one or more other components, e.g., a needle, guide wire, and/or other instrument for creating a puncture, a source of inflation media, and/or a source of additional sealing compound (not shown), for example, to provide a kit for a medical procedure.

As shown, the cartridge 16 includes an elongate tubular member 20 carrying the sealant 2 therein, an advancer tube or member 30 adjacent the sealant 2 within the tubular member 20, and a handle or hub 23 coupled to the tubular member 20. Generally, as best seen in FIG. 1, the tubular member 20 includes a proximal end 22 coupled to the hub 23, a distal end 24 sized for introduction into an introducer sheath and/or puncture (not shown), and a lumen 26 extending between proximal and distal ends 22, 24 of the tubular member 20. The tubular member 20 may be substantially rigid, semi-rigid, or flexible, e.g., such that the tubular member 20 may be advanced through an introducer sheath or otherwise into a puncture through tissue. Optionally, the hub 23 may include one or more detents or other features (not shown) for releasably coupling the cartridge 16 to the positioning member 14, e.g., as described in the references incorporated by reference herein.

The advancer member 30 may be an elongate tubular body sized to be slidably received within the lumen 26 of the tubular member 20, although the advancer member 30 may abut or otherwise interact with the hub 23 of the cartridge 16, e.g., such that the advancer member 30 is advanced distally when the cartridge 16 is advanced. A distal end 34 of the advancer member 30 may terminate in a substantially blunt distal tip proximal to the tubular member distal end 24, which may facilitate contacting and/or otherwise maintaining the sealant 2 within a puncture, e.g., when the tubular member 20 is retracted during use, as described further below.

The advancer member 30 may be substantially rigid, semi-rigid, and/or substantially flexible, e.g., having sufficient column strength to allow proximal movement of the tubular member 20 relative to the sealant 2 without buckling the advancer member 30 and/or to allow the distal end 34 of the advancer member 30 to be advanced to compress the sealant 2 within a puncture, e.g., by pushing from the proximal end 32, as described further below. The advancer member 30 may also include a lumen (not shown) extending between the proximal and distal ends 32, 34, e.g., to accommodate the positioning member 14, a flowable sealing compound, and/or fluid (not shown).

Optionally, the advancer member 30 may include one or more elements (not shown), e.g., on the proximal end, for interacting with one or more cooperating elements (also not shown) on the positioning member 14, e.g., to limit movement of the advancer member 30 relative to the positioning member 14. For example, the cooperating elements may interact to limit advancement of the advancer member 30 and/or the entire cartridge 16, e.g., to limit advancement of the sealant 2 to a predetermined offset distance relative to a positioning element 46 on the positioning member 14, as described further below.

As shown in phantom, the sealant 2 may be disposed within the lumen 26 of the tubular member 20 proximate to the distal end 24, e.g., immediately adjacent a distal tip 25 thereof or offset a predetermined distance from the distal tip 25, e.g., between about one and ten millimeters (1-10 mm) or between about one and five millimeters (1-5 mm). The lumen 26 may be sized such that the tubular member 20 and sealant 2 are slidable relative to one another, e.g., to allow the tubular member 20 to be retracted proximally relative to the sealant 2 and/or advancer member 30, as described further below.

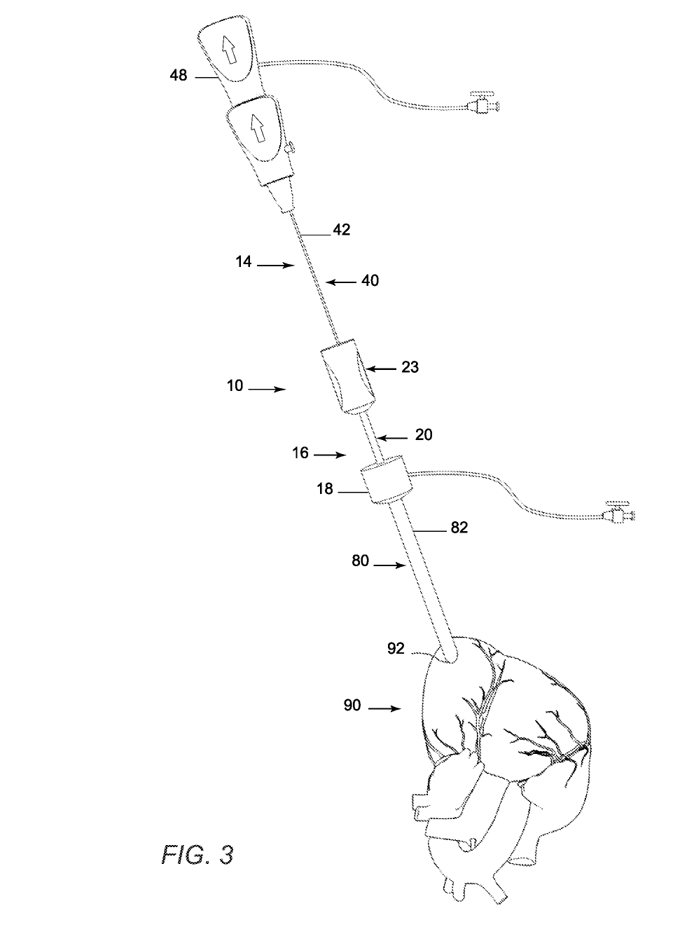

With continued reference to FIG. 1, the positioning member 14 generally includes an elongate member 40 including a proximal end 42 (not shown, see, e.g., FIG. 3), a distal end 44, and an occlusion or positioning element 46 on the distal end 44. The positioning element 46 may be an expandable member, such as a balloon, a wire mesh structure, an expandable frame, and the like, e.g., as disclosed in the references incorporated by reference herein. The positioning element 46 may be selectively expandable, e.g., using a source of inflation media, such as a syringe, a pull wire, and/or other actuator (not shown), operable from the proximal end 42 of the positioning member 14.

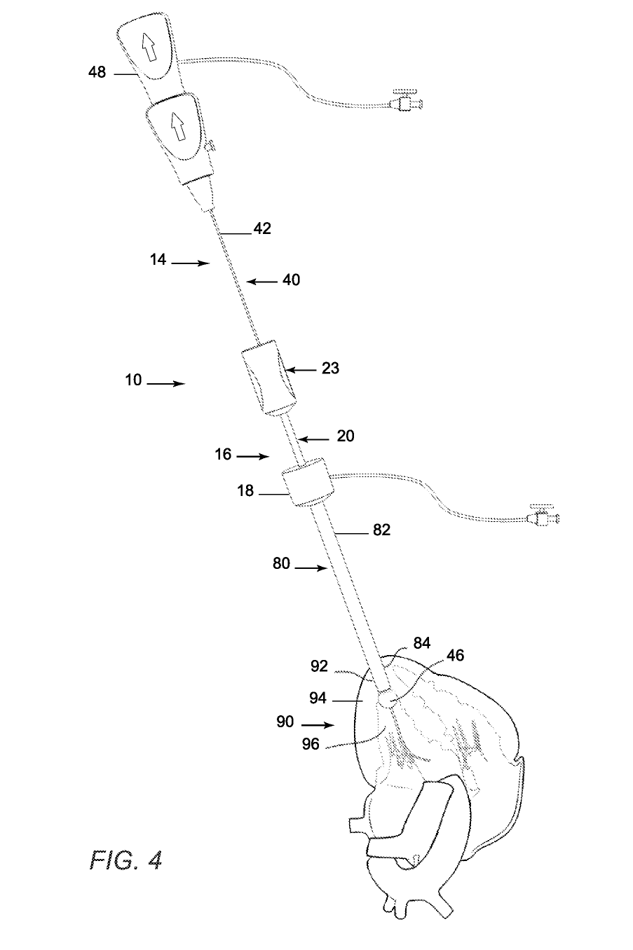

For example, as shown in FIGS. 1 and 4, the positioning element may be a balloon 46, and the positioning member 14 may include a tubular body 40 including a lumen (not shown) extending between the proximal and distal ends 42, 44 and communicating with an interior of the balloon 46. In this embodiment, the positioning member 14 may include a source of inflation media, such as syringe, that may be coupled to a housing 48 on the proximal end 42 of the positioning member 14. Optionally, the positioning member 14 may include an internal pull wire (not shown) that causes the balloon 46 to shorten during expansion and extend during collapse. Exemplary embodiments of positioning members 14 including balloons that may be used are disclosed in U.S. Publication Nos. 2004/0249342, 2004/0267308, 2006/0253072, and 2008/0009794. The entire disclosures of these references are expressly incorporated by reference herein.

Alternatively, the positioning element may be biased to an enlarged condition, but may be compressed to a contracted condition, e.g., by an overlying sleeve or other constraint (not shown). The constraint may be removed to expose the positioning element, allowing the expandable element to automatically expand to the enlarged condition. Additional information on expandable structures that may be provided on the positioning member 14 may be found in U.S. Pat. Nos. 6,238,412, 6,635,068, and 6,890.343, and in co-pending application Ser. No. 10/975,205, filed Oct. 27, 2004. The entire disclosures of these references are expressly incorporated herein by reference.

With additional reference to FIGS. 2-8, the apparatus 10 may be used to position and deliver the sealant 2 within a puncture, hole, or other opening 92, e.g., extending through the myocardium of a heart 90, as described further elsewhere herein. In one embodiment, as shown in FIGS. 1 and 2, the cartridge 16 (along with the advancer member 30 and sealant 2 within the tubular member 20) may be initially provided on the proximal end 42 of the positioning member 14. For example, the housing 48 on the positioning member 14 and the hub 23 on the cartridge 16 may be initially coupled to one another, e.g., using one or more releasable detents (not shown). Alternatively, the cartridge 16 may be initially provided such that the distal 24 of the tubular member 20 is disposed adjacent the balloon 46, similar to the distal position shown in FIGS. 3 and 4, e.g., as disclosed in U.S. Pat. No. 7,335,220 and U.S. Publication No. 2008/0082122, incorporated by reference elsewhere herein.

As shown in FIGS. 2-4, the cartridge 16 may be slidable distally along the positioning member 14, e.g., by disconnecting the hub 23 from the housing 48, and then advancing the cartridge 16, e.g., until the distal end 24 of the tubular member 20 is disposed adjacent the positioning element 46. For example, detents on the hub 23 and housing 48 may simply separate from one another when the hub 23 is advanced away from the housing 48 with sufficient force. Alternatively, one of the hub 23 and housing 48 may include an actuator or lock that may be activated (not shown) to separate the detents and/or otherwise allow the cartridge 16 to be advanced relative to the positioning member 14.

Optionally, the cartridge 16 and/or positioning member 14 may include cooperating features that limit distal movement of the cartridge 16 relative to the positioning member 14. For example, the hub 23 of the cartridge 16 may include a pocket and the positioning member 14 may include a detent or other feature (both not shown) that may be received within the pocket when the cartridge 16 is advanced to a distal position. In addition or alternatively, the positioning member 14 and/or advancer member 30 may include one or more elements that engage when the cartridge 16 reaches a predetermined location when advanced along the positioning member 14, e.g., to limit subsequent distal and/or proximal movement of the advancer member 30 relative to the positioning member 14, e.g., when the tubular member 20 is subsequently refracted, similar to embodiments disclosed in the references incorporated by reference herein.

In addition or alternatively, one or more markers may be provided on the apparatus 10, e.g., to identify when components are located at one or more desired positions or otherwise to facilitate use of the apparatus 10. For example, the positioning member 14 may include one or more markers (not shown) at predetermined locations on the elongate member 40. Such markers may provide visual confirmation when the cartridge 16 has been advanced to a desired distal position, e.g., when the marker(s) emerge from the hub 23 as the cartridge 16 is advanced over the positioning member 14. In addition or alternatively, the advancer member 30 may include one or more markers thereon (also not shown), which may be visible when the cartridge 16 is advanced to a distal position and then the tubular member 20 is retracted to expose the sealant 2. These markers may also provide visual guides to inform the user when the advancer member 30 is manipulated, e.g., advanced into a puncture to compress the sealant 2 therein, as described further below.

The apparatus 10 may be assembled using conventional manufacturing methods and/or using methods disclosed in the references incorporated by reference herein. The components of the cartridge 16, the tubular body 20, advancer tube 30, and hub 23 may be formed using conventional methods, e.g., extruding, molding, and the like. For example, the hub 23 may be formed from a plurality of molded shells that may be attached together and to which the proximal end 22 of the tubular body 20 may be attached. In the exemplary embodiment shown, the cartridge 16 includes a single tubular body 20 attached to the hub 23. In an alternative embodiment, the cartridge 16 may include inner and outer cartridge assemblies, including inner and outer tubular bodies (not shown) attached to the hub 23, e.g., similar to embodiments disclosed in the references incorporated by reference herein.

The advancer member 30 may include a section of tubing with a thermoformed tapered tip. Once the tubular body 20 (or bodies) is assembled to the hub 23, the advancer member 30 may be inserted into the lumen 26 of the tubular body 20 (e.g., into the inner cartridge tubing if inner and outer cartridge tubular bodies are provided).

To provide the hub 48 of the positioning member 14, a hub barrel (not shown), stopcock 48b, and extension line 48c may be assembled, similar to embodiments disclosed in the references incorporated by reference herein. One end of the extension line 48c may be bonded or otherwise attached to the stopcock 48b, and the other end of the extension line 48c may be bonded or otherwise attached into a side port of the hub barrel. To complete the positioning member 14, locking features (not shown) may be bonded onto the tubular body 40, e.g., spaced a predetermined distance from the proximal end 42.

The proximal leg of the balloon 46 may be bonded to the distal end 44 of the tubular body 40. The cartridge 16, hub barrel 48 and a core wire with tension plunger (not shown) are all then assembled with the tubular body 40, e.g., similar to embodiments in the references incorporated by reference herein. The core wire may then be bonded into the distal leg of the balloon 46. The hub barrel may be bonded to the proximal end 42 of the tubular body 40 and captured within the halves of a handle shell to provide the hub 48, as shown in FIG. 2.

Finally, the sealant 2 may be loaded into the assembled apparatus 10. For example, a rolled sealant 2 may be coaxially mounted over the tubular body 40 from the distal end 44 and positioned inside the tubular member 20 of the cartridge 16, e.g., adjacent the distal end 24 and the advancer member 30 therein.

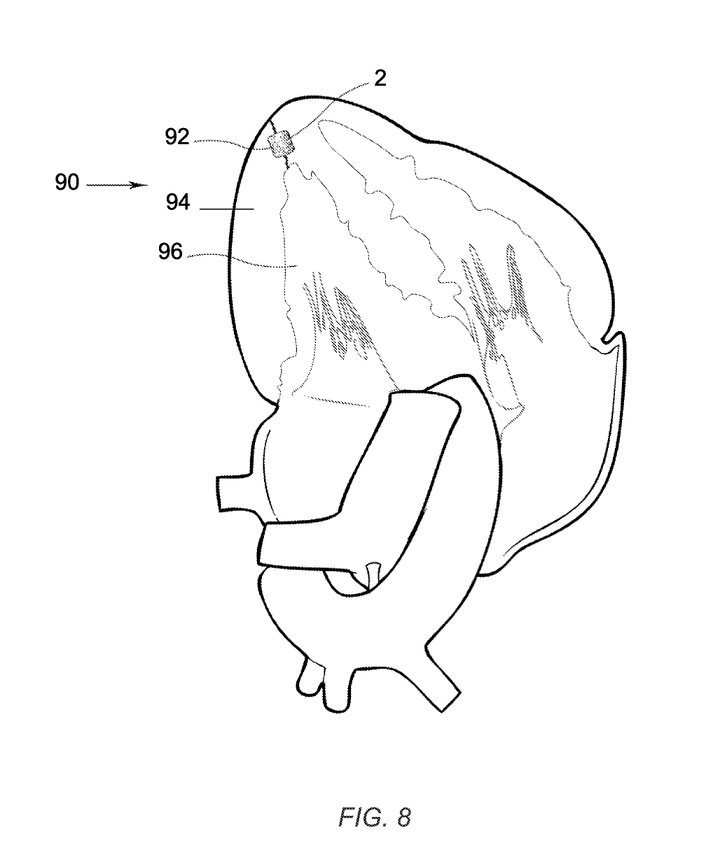

The pellet or sealant 2 may have an “as delivered” diameter ranging from much smaller than the inner diameter of the tubular member 20 to substantially the same as the inside diameter of the tubular member 20, e.g., between about one and ten millimeters (1-10 mm), at least about two millimeters (2 mm), at least about four millimeters (4 mm), at least about six millimeters (6 mm), or at least about eight millimeters (8 mm). The length of the “as delivered” sealant 2 may range from about two millimeters (2 mm) to as long as the predicted thickness of the myocardial wall, e.g., between about five and thirty millimeters (5-30 mm). Typically, the length may be designed shorter than the predicted thickness of the myocardial wall such that, after deployment, the tract on the proximal and distal ends of the sealant s may recover to a much smaller diameter than the sealant 2 and even recover to the point of achieving casual closure of the tract, e.g., as shown in FIG. 8.

The configuration of the sealant 2 within the diameters and lengths described herein may range from 100% freeze-dried polymeric material to 100% unreacted precursors of polymeric material, e.g., similar to the sealants disclosed in application Ser. No. 13/354,278, filed Jan. 19, 2012, the entire disclosure of which is expressly incorporated by reference herein. The purpose of the freeze-dried sections may be to swell with fluids to create a mechanical barrier to blood flow, while the purpose of the unreacted precursors may be to cross-link in-situ and mechanically adhere to the walls of the tract, as well as to swell and provide a mechanical barrier to blood flow, effectively anchoring the sealant 2 in place against the blood pressure within the chamber.

For example, in one embodiment, the sealant 2 may include a rolled freeze-dried sheet of polymeric material, e.g., a hydrogel material, coated on either the entire or partial outside surface with a layer of unreacted precursors. In an alternative embodiment, the sealant 2 may include a rolled freeze-dried sheet of polymeric material with a proximal and/or distal segment of unreacted precursors attached to one or both ends of the polymeric material. In further alternatives, the sealant 2 may include segments of alternating rolled freeze-dried sheet and solid unreacted precursors and/or a rolled, freeze-dried sheet with spaced bands of unreacted precursor coatings on the outside. Alternatively, in any of the embodiments herein, the “rolled, freeze-dried sheet” may be replaced with a formed cylindrical piece of freeze-dried polymer.

Additional exemplary embodiments of pellets and/or sealants that may be used are disclosed in U.S. Pat. Nos. 7,316,704, 7,331,979, 7,335,220, and 7,806,856, in U.S. Publication Nos. 2007/0231366, 2008/0082122, 2009/0088793, 2009/0254110, 2010/0168789, 2010/0274280, and 2010/0280546, and in co-pending application Ser. Nos. 13/105,822, filed May 11, 2011, and 61/434,412, filed Jan. 19, 2011. The entire disclosures of these references are expressly incorporated by reference herein.

Turning to FIGS. 2-8, an exemplary method is shown for sealing a puncture 92, e.g., using the apparatus 10 to deliver a sealant 2 (which again may be any of the exemplary embodiments herein), e.g., to achieve hemostasis within the puncture 92. As shown, the puncture 92 may be a hole or other opening created through the wall 94 of the heart 90, e.g., through the myocardium at the apex of the heart from outside the heart 90 into the left ventricle 96, as best seen in FIGS. 4-8. It will be appreciated that the opening 92 may be created through the wall of the heart 90 at other locations, e.g., to access the left ventricle 96 or other chambers of the heart 90. In a further alternative, the opening 92 may be created in other organs or tissue structures within a patient's body. The resulting opening 92 may have a diameter or other maximum cross-section, e.g., between about 5 F and 24 F (1⅔ to 8 mm).

In an exemplary method, the puncture 92 may be created using known procedures, e.g., using a needle, guide wire, one or more dilators, and the like (not shown). A distal end 84 of an introducer sheath 80 may be advanced through the puncture 92 into the left ventricle 96, e.g., over a guide wire (not shown) placed through the puncture 92 into the left ventricle 96. As shown, the introducer sheath 80 may include a proximal end 82 coupled to a hub 81 opposite the distal end 84. The introducer sheath 80 may have sufficient length to extend from within the heart 90 to outside the patient's body, e.g., via an access port, thoracotomy, sternotomy, and the like (not shown), e.g., between about ten and forty centimeters (10-40 cm).

The introducer sheath 80 may provide access into the left ventricle 96 for one or more instruments (not shown), e.g., to allow one or more diagnostic and/or interventional procedures to be performed within or via the left ventricle 96. Upon completing the procedure(s), any such instrument(s) may be removed, leaving the introducer sheath 80 extending through the puncture 92 into the left ventricle 96.

With reference to FIG. 2, the positioning member 14 may be introduced into the hub 81 and through the lumen of the introducer sheath 80, e.g., with the expandable positioning element 46 in a collapsed condition, e.g., as shown in FIG. 1. The cartridge 16, along with the sealant 2 and advancer member 30, may be provided initially on the proximal end 42 of the positioning member 40, e.g., as shown in FIGS. 1 and 2. Thus, the distal end 24 of the tubular member 20 may initially be located outside the puncture 92 when the positioning member 40 is advanced into the puncture 92, as shown in FIG. 2.

Still referring to FIG. 2, the distal end 44 of the positioning member 14 may be inserted through the puncture 92 (via the introducer sheath 80) and into the left ventricle 96. Once the positioning element 46 is disposed within the left ventricle 96, i.e., beyond a distal end 84 of the introducer sheath 80, the positioning element 46 may be expanded to an enlarged condition, e.g., as shown in FIGS. 4-6.

After expanding the positioning element 46, the positioning member 40 may be at least partially withdrawn until the positioning element 46 contacts the endocardial wall of the left ventricle 96, e.g., to provide temporary hemostasis and substantially seal the left ventricle 96 from the puncture 92. In an exemplary method, this may involve a two-step process (although it may be completed in a single substantially continuous action). First, with the positioning element 46 expanded within the left ventricle 96 beyond the distal end 84 of the introducer sheath 80, the positioning member 14 may be withdrawn until the positioning element 46 contacts the distal end 84 of the introducer sheath 80, which may provide a first tactile feedback to the user (i.e., that the positioning element 46 has contacted the introducer sheath 80, e.g., based upon the increased weight and/or resistance to proximal movement). The positioning member 14 may be withdrawn further until the positioning element 46 contacts the endocardial wall of the left ventricle 96 and resists further withdrawal, thereby providing a second tactile feedback. The introducer sheath 80 may be pulled proximally by the positioning element 46 as the positioning member 14 is withdrawn, e.g., until the distal end 84 of the introducer sheath 80 is withdrawn into the puncture 92.

Proximal tension may be applied and/or maintained on the positioning member 14 to hold the positioning element 46 against the wall of the left ventricle 96, e.g., to substantially seal the puncture 92 from the left ventricle 96 and/or prevent further removal of the positioning member 14. The proximal tension may be maintained manually or using a tensioner device (not shown) to provide temporary hemostasis, e.g., during the subsequent steps. Exemplary tension devices are disclosed in U.S. Publication No. 2004/0267308, incorporated by reference elsewhere herein.

Turning to FIGS. 3 and 4, the cartridge 16 (carrying the sealant 2) may then be advanced distally over the positioning member 14 into the puncture 92. As shown, the distal end 24 of the tubular member 20 may enter the introducer sheath 80 and be advanced towards the positioning element 46. The cartridge 16 may be advanced until a component of the cartridge 16 encounters a stop on the positioning member 14, thereby preventing further advancement of the cartridge 16 and/or spacing the sealant 2 a predetermined distance from the positioning element 46, e.g., between about one and ten millimeters (1-10 mm). For example, given the thickness of the wall 94 of the heart 90, it may be desirable to prevent the sealant 2 from being exposed within the left ventricle 96. This offset of the distal end of the sealant 2 from the interior of the heart 90 may be achieved with cooperating elements on the advancer member 30, tubular member, 20, hub 23, and/or other features of the cartridge 16 and the tubular body 40 and/or other features of the positioning member 14, as described elsewhere herein and in the references incorporated by reference herein.

Alternatively, the cartridge 16 may be advanced into the introducer sheath 80 until the distal end 24 contacts the expanded positioning element 46, which may provide tactile feedback that the cartridge 16 has been advanced sufficiently, or the sealant 2 is otherwise positioned within the puncture 92.

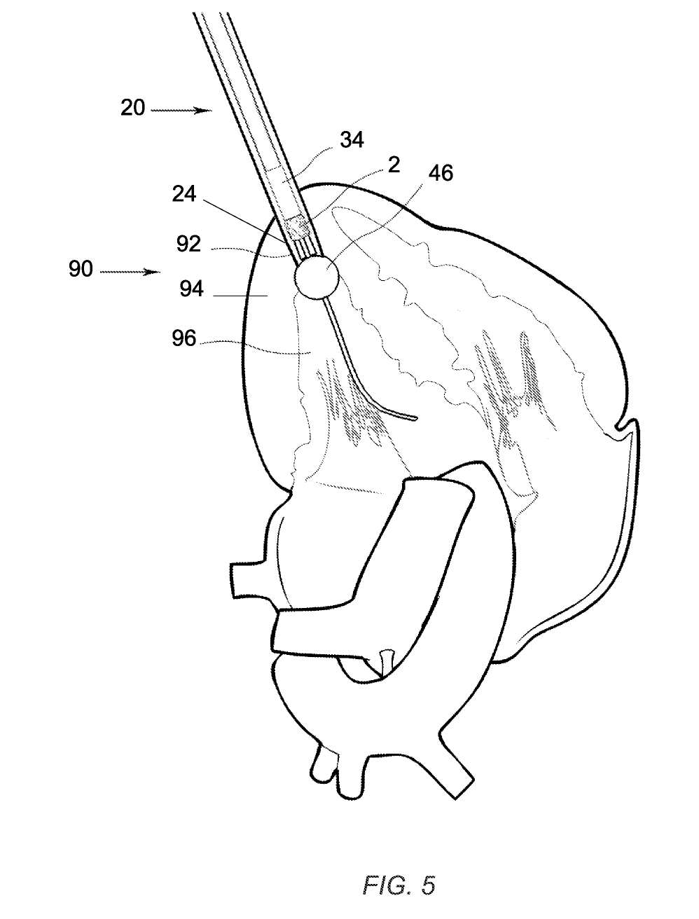

Thereafter, as shown in FIGS. 4 and 5, the tubular member 20 of the cartridge 16 and introducer sheath 80 may be retracted, e.g., by pulling proximally on the hub 81 of the introducer sheath 80, to withdrawn the introducer sheath 80 and tubular member 20 from the puncture 92 and expose the sealant 2 within the puncture 92 beyond the introducer sheath distal end 84. Optionally, a sleeve or locking device (not shown) may be provided on the cartridge 16 that may couple the introducer sheath 80 to the tubular member 20, similar to embodiments disclosed in U.S. Publication No. 2009/0088793, the entire disclosure of which is expressly incorporated by reference herein. Thus, in this alternative, if the user pulls proximally on the hub 23 or tubular member 20 rather than the hub 81 of the introducer sheath 80, the introducer sheath 80 and tubular member 20 may still be withdrawn together from the puncture 92.

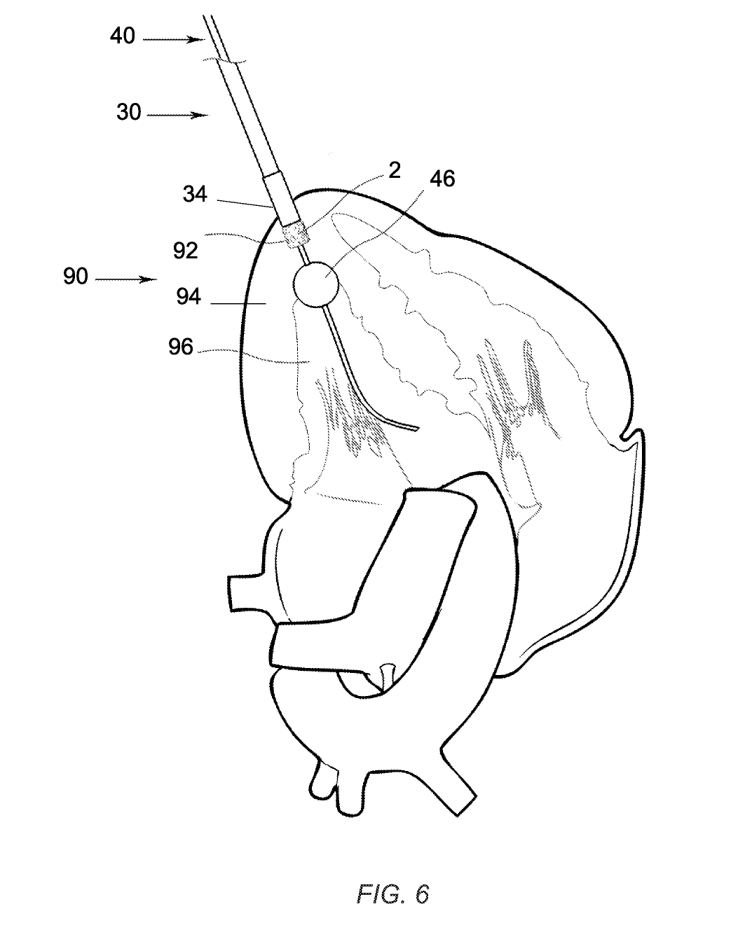

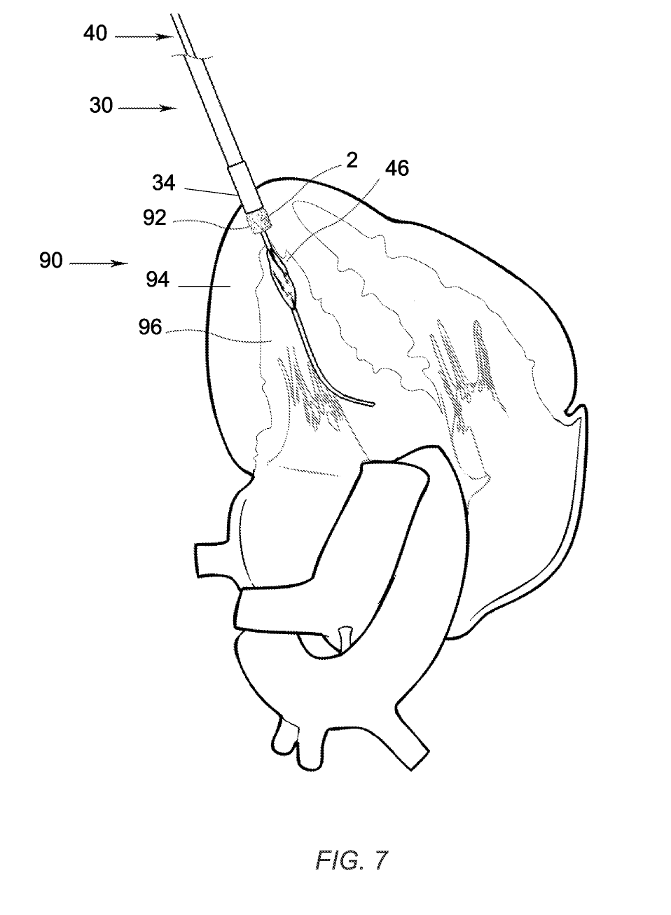

As the tubular member 20 is retracted, the advancer member 30 may prevent substantial proximal movement of the sealant 2, thereby exposing the sealant 2 within the puncture 92, as shown in FIGS. 6 and 7. For example, as described above, as the cartridge 16 is advanced, one or more features (not shown) on the proximal end 32 of the advancer member 30 may pass over a reduced region or other feature (also not shown) on the positioning member 14, thereby preventing subsequent proximal withdrawal of the advancer member 30 relative to the positioning member 14. Thus, when the cartridge 16 is then retracted, the features may prevent substantial proximal movement of the advancer member 30, and the sealant 2 adjacent the distal end 34 of the advancer member 30.

When the sealant 2 is exposed within the puncture 92, the sealant 2 may be exposed to blood and/or other body fluids within the puncture 92. This exposure may cause the sealant 2 to absorb fluid and activate to provide hemostasis, as described further elsewhere herein. Optionally, once the sealant 2 is exposed within the puncture 92, the advancer member 30 may be advanced to compress or tamp the sealant 2 (not shown), e.g., against the positioning element 46. In this option, the advancer member 30 may include one or more markers (not shown), e.g., on or adjacent the proximal end 32, and the advancer member 30 may be advanced into the puncture 92 a desired distance, which may be confirmed by monitoring the markers. In addition or alternatively, the positioning member 14 may include a second feature (not shown) over which the advancer member 30 may pass when advanced a predetermined distance. The second feature may provide an audible confirmation that the advancer member 30 has been advanced the predetermined distance (in addition or instead of the visible confirmation provided by the markers 33).

Once the sealant 2 has been exposed for sufficient time and/or tamped by the advancer member 30, the positioning element 46 may be collapsed, as shown in FIG. 7, and the positioning member 14 withdrawn from the left ventricle 96 and puncture 92, e.g., pulling the collapsed positioning element 46 through the sealant 2 and advancer member 30. The advancer member 30 may be maintained substantially stationary during withdrawal of the positioning member 14, e.g., to prevent migration and/or dislodgment of the sealant 2 within the puncture 92.

Once the positioning member 14 is completely removed, the advancer member 30 may be removed from the puncture 92, leaving the sealant 2 within the puncture 92, as shown in FIG. 8. As shown, the sealant 2 may be delivered within the myocardial wall 94, e.g., without exposing ends of the sealant 2 within the chamber of the heart 90 and/or outside the heart 90. The position of the sealant 2 may be held either by the interference caused by swelling and/or by activation of an adherent property of all or parts of the sealant, as disclosed elsewhere herein and in the references incorporate by reference herein.

Optionally, after or before removing the positioning member 14, liquid hydrogel or other sealing compound, or other material may be delivered into the puncture 92, e.g., above and/or around the sealant 2, to assist in achieving hemostasis. For example, solutions may be delivered including pH buffers and/or other compounds to activate a cross-linking reaction on all or part of the sealant, as described in different embodiments elsewhere herein and in the references incorporated by reference herein. Such material may be delivered via the lumen 36 of the advancer member 30 and/or by introducing another delivery device (not shown) into the puncture 92, e.g., after removing the advancer member 30.

The delivery device may be designed to deliver such solutions and/or materials to the internal and/or external surface of the sealant 2 causing the sealant 2 to swell. The sealant 2 and/or solutions may include formulations to activate a cross-linking reaction on all or part of the sealant, as described elsewhere herein.

It will be appreciated that elements or components shown with any embodiment herein are merely exemplary for the specific embodiment and may be used on or in combination with other embodiments disclosed herein.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the scope of the appended claims.

1. A method for performing a medical procedure, comprising:
providing access to a patient's heart;
creating a passage through the myocardium of the heart into a chamber of the heart;
introducing a distal end of an access sheath through the passage into the chamber of the heart;
performing a medical procedure via the access sheath;
introducing a sealant into the passage such that a distal end of the sealant is offset from the endocardial wall of the chamber by a predetermined offset distance; and
removing the access sheath from the heart.

2. The method of claim 1, wherein introducing the sealant comprises:
inserting a tubular cartridge carrying the sealant into the access sheath; and
withdrawing the cartridge and the access sheath to deploy the sealant within the passage such that the distal end of the sealant is offset from the endocardial wall of the chamber by the predetermined offset distance.

3. The method of claim 1, wherein the predetermined offset distance is at least about two millimeters.

4. The method of claim 3, wherein the sealant has a predetermined length such that, when the sealant is introduced into the passage, a proximal end of the sealant is offset inwardly from the outer surface of the heart by a proximal offset distance.

5. The method of claim 4, wherein the proximal offset distance is at least about two millimeters.

6. The method of claim 2, further comprising:
introducing a positioning member into the access sheath until a positioning element thereon is disposed within the chamber;
expanding the positioning element within the chamber;
withdrawing the positioning member until the expanded positioning element contacts the endocardial wall of the chamber adjacent the passage; and
wherein the cartridge is inserted into the access sheath by advancing the cartridge over the positioning member.

7. The method of claim 6, further comprising:
collapsing the positioning element after deploying the sealant within the passage; and
withdrawing the positioning member such that the collapsed positioning element passes through the sealant deployed within the passage.

8. The method of claim 6, wherein the cartridge and the positioning member include cooperating elements that limit advancement of the cartridge over the positioning member until the distal end of the sealant is offset by the predetermined offset distance.

9. The method of claim 2, wherein an advancer member within the cartridge prevents substantial proximal movement of the sealant when the cartridge is withdrawn, thereby deploying the sealant from a distal end of the cartridge.

10. The method of claim 1, wherein the sealant has a predetermined outer diameter before being introduced into the passage that is at least about two millimeters (2 mm).

11. The method of claim 1, wherein the sealant is configured such that, upon exposure to fluid within the passage, the sealant expands radially outwardly to contact tissue surrounding the passage to substantially seal the passage.

12. The method of claim 11, wherein the sealant is configured not to expand substantially along its length upon exposure to fluid within the passage.

13. The method of claim 11, wherein the sealant is configured to expand radially outwardly to a greater degree than the sealant is configured to expand along its length upon exposure to fluid within the passage.

14. The method of claim 1, wherein the sealant comprises proximal and distal sections attached to a central section, the central section formed from a freeze-dried hydrogel that expands when exposed to physiological fluid within a puncture, the proximal and distal sections formed from non-freeze-dried, non-crosslinked hydrogel precursors, the precursors remaining in an unreactive state until exposed to an aqueous physiological, whereupon the precursors undergo in-situ crosslinking with one another to provide proximal and distal adhesive layers bonded to the central section.

15. The method of claim 1, wherein the sealant comprises a cylindrical member defining an outer surface and formed from a freeze-dried hydrogel that expands when exposed to physiological fluid within a puncture, the outer surface being at least partially covered with a coating formed from non-freeze-dried, non-crosslinked hydrogel precursors, the precursors remaining in an unreactive state until exposed to an aqueous physiological, whereupon the precursors undergo in-situ crosslinking with one another to provide an outer adhesive layer bonded to and surrounding the cylindrical member.

16-18. (canceled)

19. The method of claim 1, wherein the chamber comprises the left ventricle of the heart.

20. The method of claim 19, wherein the passage is created at the apex of the heart through the myocardium into the left ventricle.

21. A method for sealing a puncture through the wall of a patient's heart, comprising:
introducing a positioning member into the puncture until a positioning element thereon is disposed within the chamber;
expanding the positioning element within the chamber;
withdrawing the positioning member until the expanded positioning element contacts the endocardial wall of the chamber adjacent the passage;
advancing a cartridge carrying a solid sealant over the positioning member; and
deploying the sealant within the puncture, the sealant expanding upon exposure to fluid within the puncture to substantially seal the puncture.

22. The method of claim 21, further comprising providing an access sheath comprising a distal end positioned through the puncture into a chamber of the heart, and a proximal end extending out of the patient's heart, and wherein the positioning member is introduced into the puncture through the access sheath.

23. The method of claim 22, wherein the cartridge is advanced over the positioning member into the access sheath until a distal end of the sealant is spaced apart from the positioning element, thereby offsetting the sealant from the endocardial wall of the chamber by a predetermined distance.

24. The method of claim 21, wherein the cartridge is advanced over the positioning member to position a distal end of the sealant spaced apart from the positioning element, thereby offsetting the sealant from the endocardial wall of the chamber by a predetermined offset distance.

25. The method of claim 24, wherein the cartridge and the positioning member include cooperating elements that limit advancement of the cartridge over the positioning member until the distal end of the sealant is offset by the predetermined offset distance.

26. The method of claim 21, wherein the sealant is configured to expand radially outwardly to a greater degree than the sealant is configured to expand along its length upon exposure to fluid within the passage.

27. The method of claim 26, wherein the sealant is configured such that, upon expansion within the passage, a distal end of the sealant does not extend into the chamber and a proximal end of the sealant does not extend out of the heart.

28. (canceled)

29. A method for sealing a puncture through the wall of a patient's heart using an apparatus comprising an elongate positioning member carrying a cartridge, the cartridge carrying a sealant and a pusher member adjacent the sealant within a lumen of the cartridge, the method comprising:
introducing a distal end of the positioning member into the puncture with the cartridge until a positioning element thereon is disposed within a chamber of the heart;
expanding the positioning element within the chamber;
withdrawing the positioning member from the puncture until the expanded positioning element contacts an endocardial wall of the chamber adjacent the puncture;
advancing the cartridge over the positioning member into the puncture until a distal end of the sealant is spaced apart from the positioning element, thereby offsetting the sealant from the chamber by a predetermined distance; and
retracting the cartridge to deploy the sealant within the puncture.

30. The method of claim 29, wherein the cartridge and the positioning member include cooperating elements that limit advancement of the cartridge over the positioning member until the distal end of the sealant is offset by the predetermined offset distance.

31. An apparatus for sealing a puncture extending through the wall of a patient's heart, comprising:
a cartridge comprising a proximal end, a distal end sized for insertion into a puncture, a lumen extending between the proximal and distal ends, and a distal opening in communication with the lumen;
solid sealant disposed within the lumen, having a predetermined outer diameter before being deployed from the cartridge that is at least about two millimeters (2 mm); and
an advancer member disposed within the lumen of the cartridge for deploying the sealant from the lumen out the distal opening when the cartridge is retracted from a puncture relative to the advancer member.

31-47. (canceled)
